(19) United States
(12) Patent Application Publication
(10) Pub. No.: US 2017/0157143 A1
(43) Pub. Date: Jun. 8, 2017

(54) NORPREGNANE DERIVATIVES FOR THE RISK REDUCTION OF BREAST CANCER

(71) Applicant: Helix Twelve Pharmaceuticals Corporation, Inc., Palo Alto, CA (US)

(72) Inventors: Masato Tanabe, Palo Alto, CA (US); Richard H. Peters, Reno, NV (US); Wan-Ru Chao, Sunnyvale, CA (US); Fred Schaufele, San Francisco, CA (US)

(73) Assignee: Helix Twelve Pharmaceuticals Corporation, Inc., Palo Alto, CA (US)

(21) Appl. No.: 15/366,836

(22) Filed: Dec. 1, 2016

Related U.S. Application Data
(60) Provisional application No. 62/262,272, filed on Dec. 2, 2015.

(51) Int. Cl. A61K 31/57; A61K 9/00

(52) U.S. Cl. CPC A61K 31/57; A61K 9/0053; A61K 9/0014

(57) ABSTRACT

In one embodiment, the application discloses a method for the prophylaxis or the treatment of a pre-cancerous lesion, including atypical intraductal breast hyperplasia, in a subject, wherein the method comprises administering to the subject a therapeutically effective amount of a pharmaceutical composition comprising compound of the formula I.

### RELATED APPLICATION

This application claims the benefit of U.S. Provisional Application No. 62/262,272 filed Dec. 2, 2015, which is incorporated herein by reference.

### FIELD OF THE INVENTION

The present invention generally relates to compounds, compositions and their methods for use in preventing proliferative disorders or the reduction in the risk of breast cancer in patients.

### BACKGROUND OF THE INVENTION

Breast cancer is the most frequently diagnosed malignant neoplasia and is a leading cause of cancer death in females worldwide. Breast cancer ranks second overall in cancer mortality (10.9%) and accounts for 23% (1.38 million) of new cancer diagnoses and 14% (458,400) of total cancer deaths. Breast cancer is not a single disease but instead constitutes a spectrum of lesions with distinct cellular origins, somatic changes and etiologies. Gene expression studies have divided breast cancer into several categories, i.e., basal-like, ErbB2-enriched, normal breast-like (adipose tissue gene signature), luminal subtype A and luminal subtype B. More than 70% of breast carcinomas express estrogen receptor alpha (ERa) and respond to antiestrogen therapies. These carcinomas may also express progesterone receptors (PRs), which are reliable markers of functional ERs.

Individuals who currently do not have breast cancer but who are deemed to be at risk for developing breast cancer can be risk-assessed with scientifically validated Breast Cancer Risk Assessment Tool, such as the Gail risk assessment model. Various studies have suggested that, in women found to be at risk for developing breast cancer (a typical indicator of ‘at risk’ being a 1.67% or higher likelihood of developing breast cancer over the next five years), anti-estrogen therapy reduces the risk by 30-50%, depending on the study. Even though drugs such as tamoxifen and some aromatase inhibitors have been approved for this risk reduction therapy for over a decade, less than 1% of at risk women take such drugs because of the troubling side effect profiles including a substantial risk of deep vein thrombembolism, stroke and uterine cancer (all for tamoxifen) and bone fractures and severe muscle aches (both for aromatase inhibition). Both drug classes also are known to induce hot flashes.

Individuals who currently do not have breast cancer may also be found upon examination to have abnormalities referred to as intraductal hyperplasias (subclassified as ‘typical’ or ‘atypical’) and more advanced precancerous lesions referred to as ‘ductal carcinoma in situ’ (DCIS). Currently, DCIS patients typically are treated and many of those with intraductal hyperplasias also opt for some form of treatment. At least for DCIS, tamoxifen treatment is known to reduce by 30% the risk to progression to breast cancer. Given that the primary current mode of treatment (surgery) which has recently been shown to be ineffective in lowering the numbers of women with prior DCIS who develop breast tumors (reported just within 3-4 months), a return to the more effective drug treatment is likely, although again impeded by the side effects of tamoxifen treatment.

Again, it is well known that use of tamoxifen for the prevention of de novo breast carcinoma entails a number of severe side effects and safety concerns such as a worsening of hot flushes, sweats, and vaginal discharge, blood clots, liver damage, endometrial cancer, and uterine sarcoma. Additionally, women who have had blood clots should not undergo tamoxifen therapy for the purpose of prevention of breast cancer and to prevent DCIS from developing into invasive breast cancer, and in reducing the occurrence of breast cancer in women who are at high risk for developing the disease.

Surprisingly, HLX-801, which does not have those side effects, is anticipated to be effective and superior to tamoxifen for prevention treatment with improved compliance with staying on treatment because of minimized side effects.

Accordingly, there is an urgent need to identify novel and effective prophylactic regimens utilizing orally active anti-proliferative compounds to prevent and to reduce risks of occurence of or progression to proliferative disorders in patients who are determined to be at risk, which can be administered in a reduced dosage, and exhibits unexpected efficacy. This, in turn, could lead to reduction or elimination of toxic side effects compared to existing prophylactic therapy, a resulting decrease in toxicity to healthy cells and in the cost of the treatment regimen.

### SUMMARY OF THE INVENTION

In one embodiment, the present application discloses that the compounds of the formulae I and Ia (HLX-801) are more effective in preventing breast cancer than the standard dose of oral tamoxifen, with fewer adverse side effects. The compounds are associated with the absence of the side effects that currently limit the adoption of the currently available tamoxifen and aromatase inhibition approaches by at risk individuals. Specifically, it is discovered that HLX-801 shows:

a) no evidence of venous thrombembolism which is a dangerous condition of itself and is considered the indicator of the extent to which the patients will be at risk for b) stroke;

c) no evidence of uterine thickening which is considered the indicator of the extent to which the patients will be at risk for developing treatment-induced uterine cancer;

d) no reports of muscle aches that cause many patients to stop treatment, for example, when using aromatase inhibitors for risk reduction; and

e) no evidence of bone loss that provides a substantial long-term risk of developing fractures.

Accordingly, a continuing need exists for compounds that can serve as monotherapy, for example, for intraductal hyperplasias and ductal carcinoma in situ and for those with no current pre-cancerous or cancerous breast lesions who, because of their history, are at elevated risk of developing breast cancer. Prophylaxis of breast cancer suggests a mode of action in which those individuals whose risk is lowered by tamoxifen and aromatase inhibition therapies would similarly benefit from HLX-801 therapy, albeit without the adverse effects profile typically associated with tamoxifen and aromatase inhibition.

The following embodiments, aspects and variations thereof are exemplary and illustrative are not intended to be limiting in scope.

In one embodiment, the application discloses a method for the prophylaxis or treatment of a pre-cancerous lesion in a subject, wherein the method comprises administering to the subject a therapeutically effective amount of a pharmaceutical composition comprising compound of the formula I

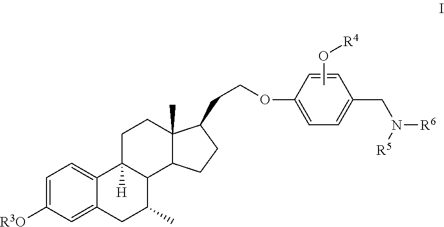

wherein R³ is H or is selected from the group consisting of C₁₋₆alkylC(O)—, C₆H₅CH₂—, C₆H₅C(O)— and —OSO₂NR′R″ where R′ and R″ are each independently H or C₁₋₃alkyl; R⁴ is H or is selected from the group consisting of C₁-₆alkyl, C₁₋₆alkylC(O)—, C₆H₅CH₂— and C₆H₅C(O)—; wherein the substituent —O—R⁴ is substituted at the 2-phenoxy or 3-phenoxy position; and R⁵ and R⁶ are each independently selected from H or the group consisting of C₁-₆alkyl, C₆H₅CH₂—, C₁₋₆alkylC(O)— and C₆H₅C(O)—; and pharmaceutically acceptable salts thereof; wherein the pre-cancerous lesion comprises atypical intraductal hyperplasia and ductal carcinoma in situ.

In one embodiment of the above method, the compound of the formula I is a compound of the formula Ia

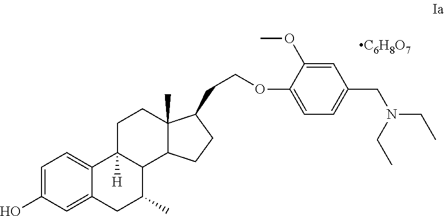

and pharmaceutically acceptable salts thereof.

In addition to the exemplary embodiments, aspects and variations described above, further embodiments, aspects and variations will become apparent by reference to the drawings and figures and by examination of the following descriptions.

The foregoing examples of the related art and limitations are intended to be illustrative and not exclusive. Other limitations of the related art will become apparent to those of skill in the art upon a reading of the specification and a study of the figures as provided herein.

### DETAILED DESCRIPTION OF THE INVENTION

### Definitions:

Unless specifically noted otherwise herein, the definitions of the terms used are standard definitions used in the art of organic synthesis and pharmaceutical sciences. Exemplary embodiments, aspects and variations are illustratived in the figures and drawings, and it is intended that the embodiments, aspects and variations, and the figures and drawings disclosed herein are to be considered illustrative and not limiting.

An “alkenyl”, alone or in combination, refers to an optionally substituted straight-chain or branched-chain hydrocarbon radical having one or more carbon-carbon double-bonds and having from 2 to about 20 carbon atoms, or from 2 to 12 carbon atoms. Examples of alkenyl radicals include ethenyl, propenyl, 1,4-butadienyl and the like.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁₋C₂₀)alkyl, for example, includes alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl, hexa-1,3,5-trienyl, and the like. An alkyl group may also be represented, for example, as a —(CR¹R²)ₘ— group where R¹ and R² are independently hydrogen or are independently absent, and for example, m is 1 to 8, and such representation is also intended to cover both saturated and unsaturated alkyl groups.

An alkyl as noted with another group such as an aryl group, represented as “arylalkyl” for example, is intended to be a straight, branched, saturated or unsaturated aliphatic divalent group with the number of atoms indicated in the alkyl group (as in (C₁₋C₂₀)alkyl, for example) and/or aryl group (as in (C₅₋C₁₄)aryl, for example) or when no atoms are indicated means a bond between the aryl and the alkyl group. Nonexclusive examples of such group include benzyl, phenethyl and the like.

“De novo” refers to the lack of transformation or metamorphosis of normal breast cells to cancerous or malignant cells. Such a transformation may occur in stages. This de novo process is in contrast to the metastasis, colonization, or spreading of already transformed or malignant cells from the primary tumor site to new locations. The method of prevention disclosed herein also relates to the administration of a compound of formula I or Ia to a subject who is at risk of developing de novo breastcancer.

“Estrogen-dependent” conditions refer to disorders or diseases that are estrogen induced or estrogen-stimulated.

“Ductal Carcinoma In Situ” or “DCIS” as used herein refers to non-invasive cancer arising from and pathologically confirmed to be confined to the terminal duct lobular units of the breast. When a subject is diagnosed with DCIS, her risk of developing invasive breast cancer in either breast may increase significantly. In addition, DCIS does recur, and some recurrences progress to invasive cancer.

“Hormone-dependent” diseases refer to diseases which substantially originate or are influenced by the presence of hormone receptors and/or hormone-dependent pathways. The diseases include, but are not limited to, breast cancer, ovarian cancer, endometrial cancer, myeloma, anovulatory infertility and meningoma.

“Intraductal hyperplasia” or “DH” refers to a benign lesion of the breast that indicates an increased risk of breast cancer. DH could be characterized as atypical DH (“ADH”). DH is characterized by cellular proliferation (hyperplasia) within one or two breast ducts and architectural abnormalities, i.e. the cells are arranged in an abnormal or atypical way.

“Method of treatment” or “therapy” or “prevention therapy” or “risk reduction therapy” refers to the method of treatment which involves the administration of the compound of the formula I or Ia, such as HLX-801, to a subject in need thereof according to the dosages and forms disclosed in this application. As used herein, “method” also refers to prophylaxis against conditions disclosed herein.

“Pharmaceutically acceptable salts” means salt compositions that is generally considered to have the desired pharmacological activity, is considered to be safe, non-toxic and is acceptable for veterinary and human pharmaceutical applications. Such salts may include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Pre-cancerous lesion” as used herein refers to normal and abnormal lesion, including proliferative lesions without atypia which slightly increase breast cancer risk, proliferative lesions with atypia which raise the risk of breast cancer. Proliferative lesions without atypia include fibroadenoma, sclerosingadenosis, multiple papillomas or papillomatosis and radial scars. Proliferative lesions with atypia include atypical lobular hyperplasia(“ALH”), lobular carcinoma-in-situ (“LCIS”) and ADH. Pre-cancerous lesion also may refer to DCIS.

“Proliferative disorder” refers to disorders characterized by the abnormal proliferation of disease cells. For example, cancer is a proliferative disorder characterized by the abnormal proliferation of tumor cells.

“Prevention of’, “prophylaxis”, or “prevent” refer to decreasing or reducing the likelihood of a patient incurring or developing a proliferative disorder, such as breastcancer. The terms also refer to the management of the factors that could lead to disease or disorder, so as to prevent the occurrence of the disease or disorder. The disease or disorder includes, but is not limited to, proliferative disorders, such as breast cancer, or estrogen receptor-mediated disorders.

“Subject” refers to a patient or a subject at risk of developing or progressing to a proliferative disorder such as cancer. The subject may be a mammalian such as a human.

“Substituted or unsubstituted” or “optionally substituted” means that a group such as, for example, alkyl, aryl, heterocyclyl, (C₁-C₈)cycloalkyl, hetrocyclyl(C₁-C₈)alkyl, aryl(C₁-C₈)alkyl, heteroaryl, heteroaryl(C₁-C₈)alkyl, and the like, unless specifically noted otherwise, may be unsubstituted or, may substituted by 1, 2 or 3 substituents selected from the group such as halo, nitro, trifluoromethyl, trifluoromethoxy, methoxy, carboxy, —NH₂, —OH, —SH, —NHCH₃, —N(CH₃)₂, —SMe, cyano and the like.

“Therapeutically effective amount” means a drug amount that elicits any of the biological effects listed in the specification.

“Treatment” refers to processes involving a slowing, interrupting, arresting, controlling, stopping, reducing, or reversing the progression or severity of a symptom, disorder, condition, or disease such as a proliferative disorder in a subject. “Treatment” also refers to prevention of the occurrence of the proliferative disorder and the underlying cause,a decreasing, or a reduction of the risk of occurrence of symptoms. For example, the present method of “treating” encompasses prevention or decreasing of the risk of the disorder in a subject at risk of developing or progressing to a proliferative disorder, such as breast cancer.

In one embodiment, the application discloses a method for the prophylaxis or treatment of a pre-cancerous lesion in a subject, wherein the method comprises administering to the subject a therapeutically effective amount of a pharmaceutical composition comprising compound of the formula I

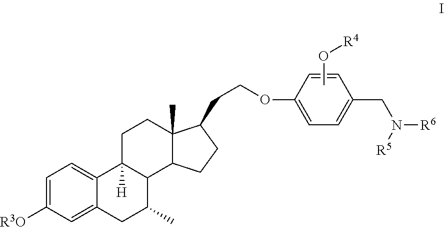

wherein R³ is H or is selected from the group consisting of C₁₋₆alkylC(O)—, C₆H₅CH₂—, C₆H₅C(O)— and —OSO₂NR′R″ where R′ and R″ are each independently H or C₁₋₃alkyl; R⁴ is H or is selected from the group consisting of C₁-₆alkyl, C₁₋₆alkylC(O)—, C₆H₅CH₂— and C₆H₅C(O)—; wherein the substituent —O—R⁴ is substituted at the 2-phenoxy or 3-phenoxy position; and R⁵ and R⁶ are each independently selected from H or the group consisting of C₁-₆alkyl, C₆H₅CH₂—, C₁₋₆alkylC(O)— and C₆H₅C(O)—; and pharmaceutically acceptable salts thereof; wherein the pre-cancerous lesion comprises atypical intraductal hyperplasia and ductal carcinoma in situ.

In one variation of the compound, R′ and R″ are both H.

In one embodiment of the above method, the compound of the formula I is a compound of the formula Ia

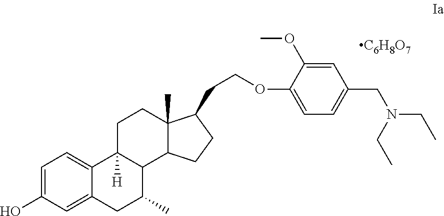

and pharmaceutically acceptable salts thereof.

In one variation, the compound of the formula Ia is (7α)-21-[4-[(diethylamino)methyl]-2-methoxyphenoxy]-7-methyl-19-norpregna-1,3,5(10)-trien-3-ol 2-hydroxy-1,2,3-propanetricarboxylate (HLX-801). The compound of the formula I and its synthesis such as, for example, HLX-801, is disclosed, for example, in U.S. Pat. No. 6,054,446, U.S. Pat. No. 6,281,205 and U.S. Pat. No. 6,455,517.

In another variation, the compound of the formula Ia is (7α)-21-[4′-(diethylamino)methyl-2′-methoxyphenoxy]-7-methyl-19-norpregna-1,3,5(10)-trien-3-ol 2-hydroxy-1,2,3-propanetricarboxylate (HLX-801). In another variation, the compound is (7α)-21-[4-[(diethylamino)methyl]-2-methoxyphenoxy]-7-methyl-19-norpregna-1,3,5(10)-trien-3-ol, or pharmaceutically acceptable salts thereof.

In one variation, the method may be used as prophylaxis against a cancer, wherein the cancer is selected from the group consisting of prostate and colon cancer, breast cancer, leukemia, renal, kidney, CNS, melanoma, lung (SCLC or NSCLC), ovarian, thyroid, advanced gastrointestinal stromal tumors (GIST) and myeloma tumor cells. In one variation, the cancer is a breast cancer.

In another embodiment of the above methods, the subject has been determined to have atypical or non-atypical intraductal hyperplasia or DCIS. In another embodiment of the method, the subject is determined to be at risk of developing atypical or non-atypical intraductal hyperplasia and ductal carcinoma in situ. In another embodiment of the method, the subject is determined to be at risk of developing atypical intraductal hyperplasia. In another embodiment, the subject is determined to be at risk of developing non-atypical intraductal hyperplasia. In yet another embodiment of the method, the subject is determined to be at risk of developing ductal carcinoma.

In one variation, the method provides an effective prophylactic treatment for pre- or postmenopausal women in preventing or reducing the risk of breast cancer occurrence or the risk of progressing to invasive breast carcinoma.

In another variation, the compound of the formula I or Ia may provide effective prophylactic treatment for endometrial carcinoma, cervix carcinoma, epithelial ovarian carcinoma, uterine (fallopian) carcinoma, fibroadenoma, macromastia (peri-postmenopausal), mastopathy, myoma and secondary or metastatic tumors derived therefrom.

In one variation, the pre-cancerous lesion may be ductal, medullary or lobular hyperplasia.

In another variation, the compounds disclosed herein may be used as prophylaxis in subjects who may be susceptible to hormone-dependent disorders, for example postmenopausal women. In another variation, the compounds may be used as prophylaxis against recurrence of pre-cancerous lesion.

In another embodiment of the above, the method is administered as a monotherapy. In another variation, the method is administered at least of one monotherapy and combination therapy for lowering the risk of breast cancer, including breast cancer de novo development, or progression to breast cancer from pre-cancerous lesions, with known or unknown risks of progression. In one variation, the compound of formula I or Ia can be combined with other pharmacologically active agents.

Many risk factors for occurrence of invasive breast cancer are well established. For instance, family and personal history of breast cancer, previous breast biopsy detection of proliferative condition such as atypical hyperplasia or DCIS, and previous breast irradiation. In one embodiment, the compounds may be administered to subjects with particular genetic risk factors including BRCA1, BRCA2, ATM, CHEK-2 and p53 mutations. In another embodiment, subjects may have certain lifestyle-related risk factors such as women who delayed childbirth until after age 30, or who had no, one or few pregnancies, or who underwent menarche at an early age, or who underwent menopause at a later age, or had long-term use of oral contraceptives, or long-term use of hormone replacement therapy. A skilled medical practitioner can evaluate these risk factors to determine whether a patient will benefit from prophylactic use of the compounds disclosed herein. In one embodiment, a practitioner may employ the Gail model to assess a subject's risk.

In another variation, the compounds disclosed herein may prove useful for preventing pre-cancerous lesion in pre-menopausal women at low dose. In another embodiment, the compounds disclosed herein are useful for lowering the risk of progression to breast cancer in women, including women with pre-cancerous lesion. In one embodiment, HLX-801 may be able to better compete for the receptors at low doses. The ability to use a low dose regime is significant in a prophylactic therapy because a subject theoretically is not exposed to the drug long-term. Additionally, any side effect associated with the compounds becomes much more tolerable.

In one variation, the compounds of formula I or Ia may be used for prophylactic indications of breast lesions. In one embodiment, the breast lesion comprises abnormal breast lesion. In another embodiment, the breast lesion comprises ductal, medullary and lobular breast lesion. In another embodiment, the compounds may be used as adjuvant or neoadjuvant therapy. Additionally, the compounds may be administered for shrinking of breast precancerous tissue, or prophylaxis against recurrence or remission of breast cancer.

In another embodiment of the above, the method comprises administering the pharmaceutical composition in a dose of from about 0.5 mg to about 80 mg, from about 1 mg to about 75 mg, from about 1.5 mg to about 70 mg, from about 2 mg to about 65 mg, from about 2.5 mg to about 60 mg, from about 3 mg to about 55 mg, from about 4 mg to about 50 mg, from about 5 mg to about 45 mg, from about 6 mg to about 40 mg, from about 7 mg to about 35 mg, from about 8 mg to about 30 mg, from about 9 mg to about 25 mg, from about 10 mg to about 20 mg, or from about 12 mg to about 15 mg.

In another embodiment, the pharmaceutical composition is administered in a fixed dose. In one variation, the composition may be administered at a fixed total dose. In another embodiment, the composition may be administered at a dose per kg body mass. In one aspect of the present application, the composition may also be administered in a dose of about 0.5 μg/kg to 10.0 μg/kg. In one embodiment, the composition may be administered in a dose of about 0.5 μg/kg, about 1.5 μg/kg, about 2.5 μg/kg, about 3.5 μg/kg, about 4.5 μg/kg, about 5.5 μg/kg, about 6.5 μg/kg, about 7.5 μg/kg, about 8.5 μg/kg, or 9.5 μg/kg. In another aspect, the composition may be administered in a daily dose of about 0.5 mg/kg, about 1.5 mg/kg, about 2.5 mg/kg, about 3.5 mg/kg, about 4.5 mg/kg, about 5.5 mg/kg, about 6.5 mg/kg, about 7.5 mg/kg, about 8.5 mg/kg, or 9.5 mg/kg. In another embodiment, the composition may be administered in a dose of about 3 μg/kg to 13 μg/kg, about 3 μg/kg to about 10 μg/kg, about 3 μg/kg to about 5 μg/kg; about 5 μg/kg to about 7 μg/kg, about 5 μg/kg to about 10 μg/kg, about 5 μg/kg to about 13 μg/kg; about 7 μg/kg to about 10 μg/kg, or about 7 μg/kg to about 13 μg/kg. In another embodiment, the composition may be administered in a dose of about 100 μg/kg to 1000 μg/kg, about 100 μg/kg to about 750 μg/kg, about 100 μg/kg to about 500 μg/kg; about 300 μg/kg to about 2000 μg/kg, about 300 μg/kg to about 1500 μg/kg, about 300 μg/kg to about 1000 μg/kg; about 500 μg/kg to about 2000 μg/kg, or about 1000 μg/kg to about 2000 μg/kg. In another embodiment, the composition may be administered in a dose of about 500 μg/kg to 2000 μg/kg, about 500 μg/kg to about 1500 μg/kg, about 500 μg/kg to about 1000 μg/kg; about 1000 μg/kg to about 2000 μg/kg, about 1000 μg/kg to about 1500 μg/kg, about 1000 μg/kg to about 2000 μg/kg; about 1500 μg/kg to about 2000 μg/kg, or about 1700 μg/kg to about 2000 μg/kg.

In another embodiment of the above method, the pharmaceutical composition is administered once daily, twice daily, three times daily, once every 2 days, once every 3 days, once every 4 days, once every 5 days, once every 6 days, once every 7 days, once every 14 days, or once every 30 days.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2 or 1 day. In some embodiments, the interval between each administration is constant. In some embodiments, the administration can be carried out daily, every two days, every three days, every four days, every five days, or weekly. In some embodiments, the administration can be carried out twice daily, three times daily, or more frequent. Administration can also be continuous and adjusted to maintaining a level of the compound within any desired and specified range.

In one variation, the administration of the composition can be extended over an extended period of time, such as from about a month or shorter up to about three years, 4 years, 5 years, 6 years or longer. For example, the dosing regimen can be extended over a period of any of about 2, 3, 4, 5, 6, 7, 8, 9, 10, 11, 12, 18, 24, 30 and 36 months. In some embodiments, there is no break in the dosing schedule. In some embodiments, the interval between each administration is no more than about a week. The compounds used in the present invention may be administered individually, or in combination with or concurrently with one or more other compounds used in other embodiments of the present invention. Additionally, compounds used in the present application may be administered in combination with or concurrently with other prophylactic agents for proliferative disorders.

In various embodiments, the pharmaceutically effective amount varies within a broad range and depends on the condition to be treated and the mode of administration. It can cover any amount efficient for the intended treatment. Determining a “pharmaceutically effective amount” is within the expertise and knowledge of the person skilled in the art.

In various embodiments, the pharmaceutically effective dose range is informed by studies conducted in mouse models in which pre-treatment for two weeks with 5, 10, 20 or 30 mg/kg/day HLX-801 results in reducing the growth of a tumor over a four-week period (by50%, 60%, 70%, 80%, 90% or 100% compared to sham-treated controls, i.e., treated in an identical manner but in the absence of HLX-801, in which 100% reduction is no subsequent tumor growth) formed after subcutaneous injection under the skin of female mice of a defined number (500,000, 1,000,000, 2,000,000 or 4,000,000) of breast tumor cells.

In another embodiment of the above method, the dosing levels established in the mouse xenograft studies, in which the mice also are co-treated with a defined amount (0.1 to 1.0 mg) of estradiol implanted into each mouse as a time-released pellet, are not the exact same doses that will be effective in humans but are partially informative of the effective dose in humans. In one aspect of the method, the mice may be co-treated with estradiol at about 0.01 to 0.5 mg/pellet, 0.01 to 0.25 mg/pellet, 0.01 to 0.1 mg/pellet, 0.1 to 0.5 mg/pellet, 0.1 to 0.25 mg/pellet, 0.1 to 0.15 mg/pellet, 0.3 to 0.5 mg/pellet, 0.3 to 0.25 mg/pellet, 0.3 to 0.15 mg/pellet, 0.5 to 0.7 mg/pellet, 0.5 to 0.8 mg/pellet, 0.5 to 0.9 mg/pellet, 0.7 to 0.8 mg/pellet, 0.7 to 0.9 mg/pellet, 0.7 to 1.0 mg/pellet, or 0.1 to 1.0 mg/pellet. In one aspect of the method, the mice may be co-treated with estradiol at about 0.01 to 1.5 mg/pellet, 0.01 to 2 mg/pellet or about 0.01 to 3 mg/pellet. In one aspect of the disclosed method, the method provides a dose of estradiol sufficient to normalize all mice throughout the studies to a similar amount of estradiol above that which they produce from endogenous synthesis.

One of skill in the art would appreciate that an effective dose of the compound of the formula I, Ia or HLX-801 administered in the animal model is typically in relationship to the amount of estradiol provided. That is, since estradiol is provided to ensure a constant amount of estradiol across all experimental animals that, because they are female and because they are cycling, will have large variations in estradiol levels throughout the course of the study. The addition of estradiol to the animals overcomes this variable, but it is one reason why the doses employed in the animal studies may be significantly different than the doses that would be calculated and used in humans in whom no excess estradiol is provided.

In one aspect, the pharmaceutically therapeutically active does of the compound of the formula I, Ia or HLX-801 may be typically formulated and administered in unit-dosage forms or multiple-dosage forms. Unit-dose forms, as used herein, refers to physically discrete units suitable for human and animal subjects and may be packaged individually as is known in the art. In one variation, each unit-dose contains a predetermined quantity of the HLX-801 sufficient to produce the desired therapeutic effect, in association with the required pharmaceutical carrier, vehicle or diluent. The concentration of the compound of the formula I, Ia or HLX-801 in the formulation may be adjusted so that the administration of a particular dose of the pharmaceutically effective amount will be sufficient to produce the desired pharmacological effect. The exact concentration of formula I, Ia or HLX-801 and/or dosage to be used will ultimately depend on the age, weight and condition of the patient or animal as is known in the art.

In another embodiment of the above method, the composition is administered orally.

In another variation of the present invention, the method of prevention may provide an improved efficacy profile, wherein the method may provide greater in-vivo activity than TAM or an aromatase inhibitor and may significantly inhibit growth of tamoxifen-resistant pre-cancerous lesion.

In one variation, the compounds disclosed herein may be active against high-risk cell lines with reduced sensitivity to TAM, fulvestrant, orraloxifene. In another embodiment, the efficacy profile of the treatment provides at least 5%, at least 10%, at least 15%, at least 20%, at least 25%, at least 30%, at least 35% or at least 40% improvement over existing prevention treatment. In other embodiments, the method may provide at least 50%, at least 60% or at least 70% improved efficacy profile over prevention treatments which do not include the compounds of formula I or Ia.

In one variation of the present application, the pharmaceutical composition comprises pharmaceutically acceptable excipients, adjuvants, diluents, additives, solvents, carriers and colorants. In another aspect, the composition is adapted for oral administration; or as a liquid formulation adapted for parenteral administration. In another aspect, the composition is adapted for administration by a route selected from the group consisting of orally, parenterally, intraperitoneally, intravenously, intra-arterial, transdermally, intramuscularly, rectally, intranasally, liposomally, subcutaneously and intrathecally.

In one variation, pharmaceutical compositions of this invention, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are suitable for parenteral administration but may also be used for oral administration. Excipients, such as polyvinylpyrrolidinone, gelatin, hydroxycellulose, acacia, polyethylene glycol, mannitol, sodium chloride or sodium citrate, may also be added. Alternatively, the compounds disclosed in the present application may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols or water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

In one variation, the pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation will be in the form of a syrup, elixir, emulsion, or an aqueous or non-aqueous suspension. Such a liquid formulation may be administered directly p.o. or filled into a soft gelatin capsule. In another embodiment of the above method, the composition is administered topically.

In one variation, the compounds may be formulated for topical administration where the dosage varies depending on patient characteristics and administration route. In one embodiment, the compound is formulated as an ointment, cream, gel, emulsion or lotion. In another embodiment, formulation may be a powder or oil. Formulation bases are familiar to the person skilled in the art from the cosmetic and pharmaceutical industry. In another embodiment, formulations may comprise vegetable oils and fats such as almond oil, peanut oil, olive oil, peach kernel oil, castor oil, plant extracts, ethereal oils. In yet another embodiment, vegetable waxes and synthetic and animal oils, fats or waxes, lecithin, lanolin alcohols, carotene, fragrances, mono- or polyhydric alcohols, urea, preservatives and coloring agents. In one embodiment, formulation as an emulsion may be contemplated.

Also included in the above embodiments, aspects and variations are salts of amino acids such as arginate and the like, gluconate, and galacturonate. Some of the compounds of the invention may form inner salts or Zwitterions. Certain of the compounds of the present invention can exist in unsolvated forms as well as solvated forms, including hydrated forms, and are intended to be within the scope of the present invention. Certain of the above compounds may also exist in one or more solid or crystalline phases or polymorphs, the variable biological activities of such polymorphs or mixtures of such polymorphs are also included in the scope of this invention. Also provided are pharmaceutical compositions comprising pharmaceutically acceptable excipients and a therapeutically effective amount of at least one compound of this invention.

Pharmaceutical compositions of the compounds of this invention, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration but may also be used for oral administration. Excipients, such as polyvinylpyrrolidinone, gelatin, hydroxycellulose, acacia, polyethylene glycol, mannitol, sodium chloride, or sodium citrate, may also be added.

Alternatively, these compounds may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols, or water. Solid carriers include starch, lactose, calcium sulfate, dihydrate, terra alba, magnesium stearate or stearic acid, talc, pectin, acacia, agar, or gelatin. The carrier may also include a sustained release material such as glyceryl monostearate or glyceryl distearate, alone or with a wax. The amount of solid carrier varies can vary between about 20 mg to about 1 g per dosage unit.

The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation will be in the form of a syrup, elixir, emulsion, or an aqueous or non-aqueous suspension. Such a liquid formulation may be administered directly p.o. or filled into a soft gelatin capsule. Suitable formulations for each of these methods of administration may be found in, for example, Remington: The Science and Practice of Pharmacy, A. Gennaro, ed., 20th edition, Lippincott, Williams & Wilkins, Philadelphia, Pa.

In one variation, there is provided the above compound, or a pharmaceutically acceptable salt thereof, optionally in the form of a single stereoisomer or mixture of stereoisomers thereof. Synthesis of HLX-801:

The compound of the formula I and Ia and its synthesis are disclosed, for example, in U.S. Pat. Nos. 6,054,446; 6,281,205 and 6,455,517. The following procedures may be employed for the preparation of the compounds of the present invention. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy, and carboxy groups are described in Greene et al., Protective Groups in Organic Synthesis, Second Edition, John Wiley and Sons, New York, 1991.Standard organic chemical reactions can be achieved by using a number of different reagents, for examples, as described in Larock: Comprehensive Organic Transformations, VCH Publishers, New York, 1989.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. The entire disclosures of all documents cited throughout this application are incorporated herein by reference.

Table 1: Exemplary effect of HLX-801 prophylactic therapy against breast cancer cells in at-risk subject groups compared to Tamoxifen (“TAM”) 1 mg.

TABLE 1

| Patients group (# of cohorts/# | TAM (mg) | HLX-801 (mg) | | | | | |
|---|---|---|---|---|---|---|---|
| of subjects per cohort) | 1 | 1 | 5 | 10 | 20 | 40 | 80 |

No. of subjects developing breast carcinoma

| | | | | | | | |
|---|---|---|---|---|---|---|---|
| 7/24 | 12 | 3 | 2 | 1 | 1 | 1 | 1 |
| 7/21 | 11 | 2 | 2 | 1 | 1 | 1 | 1 |
| 7/17 | 12 | 3 | 1 | 1 | 1 | 1 | 1 |

What is claimed is:

1. A method for the prophylaxis or the reduction in the risk of developing breast cancer or the treatment of a pre-cancerous lesion in a subject, wherein the method comprises administering to the subject a therapeutically effective amount of a pharmaceutical composition comprising compound of the formula I: 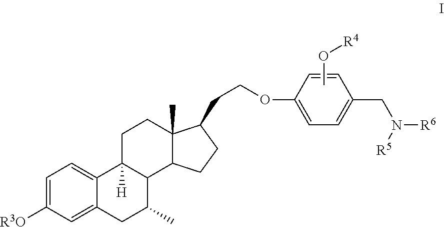
wherein R³ is H or is selected from the group consisting of C₁₋₆alkylC(O)—, C₆H₅CH₂—, C₆H₅C(O)— and —OSO₂NR′R′ where R′ and R″ are each independently H or C₁₋₃alkyl; R⁴ is H or is selected from the group consisting of C₁-₆alkyl, C₁₋₆alkylC(O)—, C₆H₅CH₂— and C₆H₅C(O)—;
wherein the substituent -O-R⁴ is substituted at the 2-phenoxy or 3-phenoxy position; and R⁵ and R⁶ are each independently selected from H or the group consisting of C₁-₆alkyl, C₆H₅CH₂-, C_{1— 6}alkylC(O)- and C₆H₅C(O)-; and pharmaceutically acceptable salts thereof;
wherein the pre-cancerous lesion comprises atypical intraductal hyperplasia.

2. The method of claim 1, wherein the pre-cancerous lesion comprises ductal carcinoma in situ.

3. The method of claim 1, wherein the pre-cancerous lesion comprises non-atypical intraductal hyperplasia.

4. The method of claim 3, wherein the compound of the formula I is a compound of the formula Ia 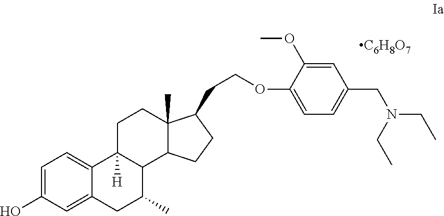
and pharmaceutically acceptable salts thereof.

5. The method of claim 4, wherein the subject is determined to be at risk of developing at least one of atypical intraductal hyperplasia and ductal carcinoma in situ.

6. The method of claim 5, wherein the method is administered as a monotherapy.

7. The method of claim 6, wherein the method comprises administering the pharmaceutical composition in a dose of from about 0.5 mg to about 80 mg, from about 1 mg to about 75 mg, from about 1.5 mg to about 70 mg, from about 2 mg to about 65 mg, from about 2.5 mg to about 60 mg, from about 3 mg to about 55 mg, from about 4 mg to about 50 mg, from about 5 mg to about 45 mg, from about 6 mg to about 40 mg, from about 7 mg to about 35 mg, from about 8 mg to about 30 mg, from about 9 mg to about 25 mg, from about 10 mg to about 20 mg, or from about 12 mg to about 15 mg.

8. The method of claim 7, wherein the pharmaceutical composition is administered in a fixed dose.

9. The method of claim 8, wherein the pharmaceutical composition is administered once daily, twice daily, three times daily, once every 2 days, once every 3 days, once every 4 days, once every 5 days, once every 6 days, once every 7 days, once every 14 days, or once every 30 days.

10. The method of claim 9, wherein the composition is administered orally.

11. The method of claim 10, wherein the composition is administered topically.
